(19) United States
(12) Patent Application Publication
(10) Pub. No.: US 2026/0195891 A1
(43) Pub. Date: Jul. 9, 2026

(54) IMAGE PROCESSING APPARATUS FOR ENDOSCOPIC EXAMINATION, IMAGE PROCESSING METHOD FOR ENDOSCOPIC EXAMINATION, AND RECORDING MEDIUM

(71) Applicant: NEC Corporation, Tokyo (JP)

(72) Inventor: Ryosaku SHINO, Tokyo (JP)

(73) Assignee: NEC Corporation, Tokyo (JP)

(21) Appl. No.: 19/131,581

(22) PCT Filed: Dec. 5, 2022
(86) PCT No.: PCT/JP2022/044683

§ 371 Date: May 21, 2025

(51) Int. Cl. G06T 7/00; A61B 1/00; A61B 5/00; G16H 50/20

(52) U.S. Cl. CPC G06T 7/0012; A61B 1/0002; A61B 5/7275; G16H 50/20; G06T 2207/10068; G06T 2207/20081; G06T 2207/30096

(57) ABSTRACT

In an information processing system, an image acquisition means acquires endoscopic images captured by an endoscope. A lesion diagnosis means diagnoses a lesion based on the endoscopic images. A surgical procedure inference means infers a surgical procedure which is recommended based on the endoscopic images and diagnosis information of the lesion. A prognosis inference means infers a state of a prognosis based on the endoscopic images and information of the surgical procedure. An output means outputs the diagnosis information, the information of the surgical procedure, and the state of the prognosis. The information processing system enables support for decision-making of medical professionals, such as doctors.

### TECHNICAL FIELD

The present disclosure relates to an inference process for a lesion in an endoscopic examination.

### BACKGROUND ART

Though it is necessary to make a plan of a treatment in a case where a lesion is discovered during an endoscopic examination, it has been difficult to make the plan of the treatment considering a prognosis, because the progress (after the treatment) is not known. Patent document 1 describes a method of proposing a surgical procedure for a joint using a trained model that has been trained on a relationship between the surgical procedure performed on the joint and a state of the joint after the surgery.

### PRECEDING TECHNICAL REFERENCES

### Patent Document

Patent Document 1: Japanese Laid-open Patent Publication No. 2021-115188

### SUMMARY

### Problem to be Solved by the Invention

It is not always possible to make appropriate recommendations regarding a procedure and prognosis with respect to a lesion found by an endoscopic examination.

It is one object of the present disclosure to provide an information processing apparatus capable of estimating recommended surgical procedures and prognosis for the lesion found by the endoscopic examination.

### Means for Solving the Problem

According to an example aspect of the present disclosure, there is provided an information processing apparatus including:
an image acquisition means configured to acquire endoscopic images captured by an endoscope;
a lesion diagnosis means configured to diagnose a lesion based on the endoscopic images;
a surgical procedure inference means configured to infer a surgical procedure which is recommended based on the endoscopic images and diagnosis information of the lesion;
a prognosis inference means configured to infer a state of a prognosis based on the endoscopic images and information of the surgical procedure; and
an output means configured to output the diagnosis information, the information of the surgical procedure, and the state of the prognosis.

According to another example aspect of the present disclosure, there is provided an information processing method including:
acquiring endoscopic images captured by an endoscope;
diagnosing a lesion based on the endoscopic images;
inferring a surgical procedure which is recommended based on the endoscopic images and diagnosis information of the lesion;
inferring a state of a prognosis based on the endoscopic images and information of the surgical procedure; and
outputting the diagnosis information, the information of the surgical procedure, and the state of the prognosis.

According to a further example aspect of the present disclosure, there is provided a recording medium storing a program, the program causing a computer to perform a process including:
acquiring endoscopic images captured by an endoscope;
diagnosing a lesion based on the endoscopic images;
inferring a surgical procedure which is recommended based on the endoscopic images and diagnosis information of the lesion;
inferring a state of a prognosis based on the endoscopic images and information of the surgical procedure; and
outputting the diagnosis information, the information of the surgical procedure, and the state of the prognosis.

### Effect of the Invention

According to the present disclosure, it becomes possible to estimate a recommended surgical procedure and prognosis for a lesion found by an endoscopic examination.

### BRIEF DESCRIPTION OF THE DRAWINGS

FIG. 1 is a block diagram illustrating a schematic structure of an endoscopic examination system.

FIG. 2 is a block diagram illustrating a hardware configuration of an information processing apparatus.

FIG. 3 is a block diagram illustrating a functional configuration of an information processing apparatus.

FIG. 4A illustrates a learning method of a surgical procedure inference model and FIG. 4B illustrates input and output data of the surgical procedure inference model.

FIG. 5A illustrates a learning method of a prognosis inference model and FIG. 5B illustrates input and output data of the prognosis inference model.

FIG. 6A illustrates a learning method of a surgical procedure and prognosis inference model and FIG. 6B illustrates input and output data of the surgical procedure and prognosis inference model.

FIG. 7 illustrates a display example by a display device.

FIG. 8 illustrates another display example by the display device.

FIG. 9 illustrates a still another display example by the display device.

FIG. 10 illustrates a further display example by the display device.

FIG. 11 is a flowchart of a data output process by the information processing apparatus.

FIG. 12 is a block diagram illustrating a functional configuration of a modification 2 of a first example embodiment.

FIG. 13 illustrates an example of a data structure of patient information.

FIG. 14 is a block diagram illustrating a functional configuration of an information processing apparatus of a second example embodiment.

FIG. 15 is a flowchart of a process by the information processing apparatus of the second example embodiment.

### EXAMPLE EMBODIMENTS

In the following, example embodiments will be described with reference to the accompanying drawings.

### First Example Embodiment

### [System Configuration]

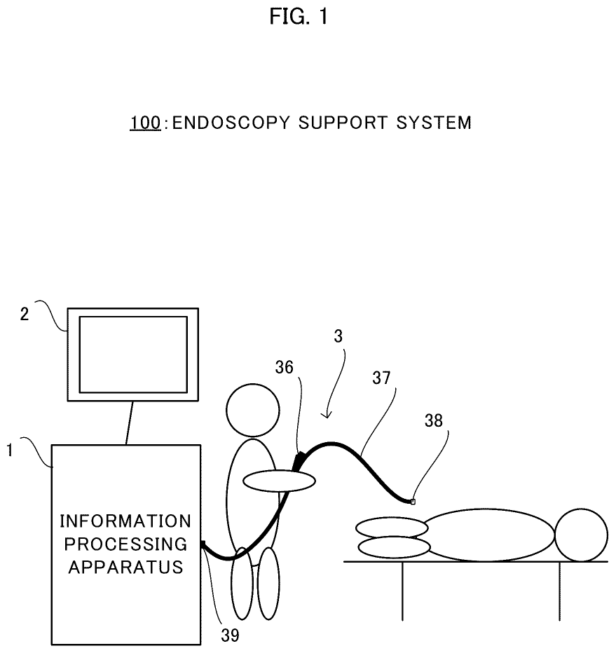

FIG. 1 shows a schematic configuration of an endoscopic examination system 100. Upon detecting a lesion during an examination (including treatment) using an endoscope, an endoscopic examination system 100 proposes a surgical procedure for the lesion, and predicts the prognosis in a case where the surgical procedure is adopted. Accordingly, it is possible for a doctor to plan the procedure while considering the prognosis.

As shown in FIG. 1, the endoscopic examination system 100 mainly includes an information processing apparatus 1, a display device 2, and an endoscope 3 which is connected to the information processing apparatus 1.

The information processing apparatus 1 acquires an image that is captured by the endoscope 3 during an endoscopic examination (i.e., a video; hereinafter, also referred to as an “endoscopic video Ic”) from the endoscope 3 and displays display data for confirmation by an examiner (doctor) of the endoscopic examination on the display device 2. Specifically, the information processing apparatus 1 acquires the video in an organ captured by the endoscope 3 as the endoscopic video Ic during the endoscopic examination. In addition, the doctor operates the endoscope 3 to input a photographing instruction at a lesion site, in a case where the lesion is found during the endoscopic examination. Based on the photographing instruction by the doctor, the information processing apparatus 1 generates one or more lesion images photographed at the lesion site. Specifically, based on the endoscopic video Ic which is video data, the information processing apparatus 1 generates lesion images, each of which is a still image, in accordance with the photographing instruction of the doctor.

The display device 2 is a display or the like for performing a predetermined display based on each display signal supplied from the information processing apparatus 1.

The endoscope 3 mainly includes an operation unit 36 for the doctor to perform inputs such as insufflation, water delivery, angle adjustment, and the photographing instruction, a shaft 37 which is to be inserted into an organ of a subject to be examined and has flexibility, a tip portion 38 incorporating an imaging unit such as an ultra-compact imaging element, and a connection unit 39 for connecting with the information processing apparatus 1.

Hereinafter, explanations will be given mainly on the assumption of processing in colonoscopy; however, an examination target is not limited to a colon and may also include a stomach, esophagus, small intestine, duodenum, and other gastrointestinal tracts (digestive organs).

### [Hardware Configuration]

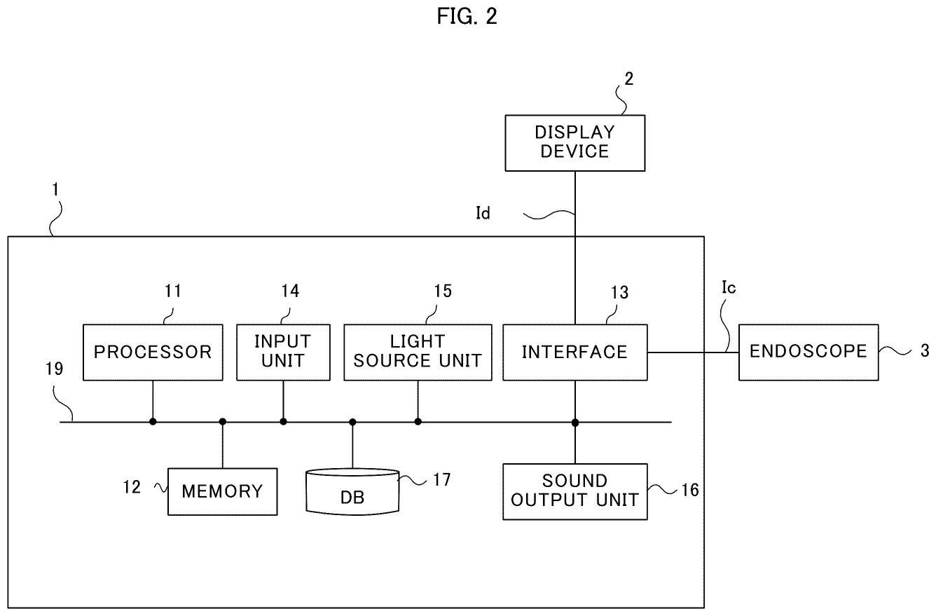

FIG. 2 shows a hardware configuration of the information processing apparatus 1. The information processing apparatus 1 mainly includes a processor 11, a memory 12, an interface 13, an input unit 14, a light source unit 15, a sound output unit 16, and a database (hereinafter, referred to as a “DB”) 17. Each of these elements is connected via a data bus 19.

The processor 11 executes a predetermined process by executing a corresponding program or the like stored in the memory 12. The processor 11 is a processor such as a CPU (Central Processing Unit), a GPU (Graphics Processing Unit), or a TPU (Tensor Processing Unit). The processor 11 may be formed by a plurality of processors. The processor 11 is an example of a computer.

The memory 12 is composed of various volatile memories used as working memories such as a RAM (Random Access Memory) and a ROM (Read Only Memory) and non-volatile memories for storing information necessary for processing the information processing apparatus 1. The memory 12 may include an external storage device, such as a hard disk, that is connected or embedded in the information processing apparatus 1, and may include a removable flash memory or a storage medium, such as a disk medium. In the memory 12, respective programs for executing processes in the present example embodiment are stored in the information processing apparatus 1.

The memory 12 also temporarily stores a sequence of the endoscopic video Ic captured by the endoscope 3 in the endoscopic examination, based on a control of the processor 11. Moreover, the memory 12 temporarily stores lesion images captured based on the photographing instruction of the doctor during the endoscopic examination. These lesion images are stored in the memory 12 in association with, for instance, subject identification information (e.g., a patient ID) and time stamp information, etc.

The interface 13 performs an interface operation between the information processing apparatus 1 and the external device. For instance, the interface 13 supplies the display data Id generated by the processor 11 to the display device 2. The interface 13 also supplies illumination light generated by the light source unit 15 to the endoscope 3. The interface 13 also provides electrical signals to the processor 11 indicative of the endoscopic video Ic supplied from the endoscope 3. The interface 13 may be a communication interface such as a network adapter for wired or wireless communication with an external device, or may be a hardware interface compliant with a USB (Universal Serial Bus), a SATA (Serial AT Attachment), etc.

The input unit 14 generates an input signal based on each operation of the doctor. The input unit 14 is, for instance, a button, a touch panel, a remote controller, a voice input device, or the like. The light source unit 15 generates light for delivery to tip 38 of endoscope 3. The light source unit 15 may also incorporate a pump or the like for delivering water or air to be supplied to the endoscope 3. The sound output unit 16 outputs sound based on the control of the processor 11.

The DB 17 stores medical record information (hereinafter, referred to as “patient information”) of the subject. Moreover, the DB 17 stores the endoscopic images and the lesion information which are acquired by a previous endoscopic examination of the subject. The lesion information includes one or more lesion images, and information related to the lesion (hereinafter referred to as “relevant information”). The DB 17 may include an external storage device, such as a hard disk, that is connected or embedded in the information processing apparatus 1, and may include a storage medium, such as a removable flash memory. Instead of providing the DB 17 in the endoscopic examination system 100, the DB 17 may be provided in an external server or the like to acquire the relevant information from the server via communications.

### [Functional Configuration]

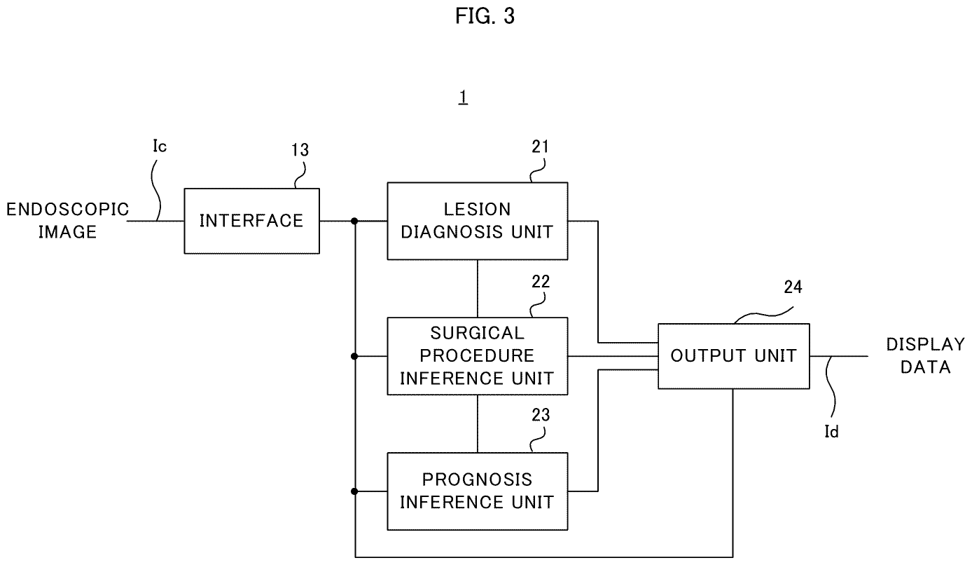

FIG. 3 is a block diagram illustrating a functional configuration of the information processing apparatus 1. The information processing apparatus 1 functionally includes a lesion diagnosis unit 21, a surgical procedure inference unit 22, a prognosis inference unit 23, and an output unit 24, in addition to the above-described interface 13.

The endoscopic video Ic is input from the endoscope 3 to the information processing apparatus 1. The endoscopic video Ic is input to the interface 13. The interface 13 extracts each frame image (hereinafter, also referred to as an “endoscopic image”) from the input endoscopic video Ic, and outputs each extracted frame image to the lesion diagnosis unit 21, the surgical procedure inference unit 22, and the prognosis inference unit 23. The interface 13 outputs the input endoscopic video Ic to the output unit 24.

The lesion diagnosis unit 21 detects the lesion based on endoscopic images input from the interface 13, and diagnoses the lesion. Specifically, the lesion diagnosis unit 21 detects the lesion based on the endoscopic images by using an image recognition model prepared in advance, and diagnoses the lesion. This image recognition model is a machine learning model which is trained in advance to detect and diagnose the lesion included in the endoscopic images, and is also called a “lesion diagnosis model” hereafter. Note that “diagnosing the lesion” refers to estimating the site of the lesion, a degree of progression of the lesion, a degree of invasion of the lesion, etc. Upon detecting the lesion, the lesion diagnosis unit 21 outputs information such as a time stamp and diagnostic information to the surgical procedure inference unit 22 and the output unit 24.

The surgical procedure inference unit 22 estimates a surgical procedure to be recommended (hereinafter, also referred to as a “recommended surgical procedure”) based on the endoscopic images input from the interface 13 and the diagnostic information input from the lesion diagnosis unit 21. Specifically, the surgical procedure inference unit 22 estimates the recommended surgical procedure based on the endoscopic images and the diagnostic information using a surgical procedure inference model which will be described later. The surgical procedure inference unit 22 outputs information of the time stamp or the like, and the recommended surgical procedure to the prognosis inference unit 23 and the output unit 24.

The prognosis inference unit 23 estimates the prognosis based on the endoscopic images input from the interface 13 and the recommended surgical procedure input from the surgical procedure inference unit 22. Specifically, the prognosis inference unit 23 estimates the prognosis based on the endoscopic images and the recommended surgical procedure using a prognosis inference model which will be described later. The prognosis includes, for instance, information such as a five-year survival rate, a duration of outpatient treatment, a dietary restriction, and use of a stoma. The prognosis inference unit 23 outputs information of the time stamp or the like and the prognosis to the output unit 24.

The output unit 24 generates the display data based on the endoscopic video Ic input from the interface 13, the diagnostic information input from the lesion diagnosis unit 21, the recommended surgical procedure input from the surgical procedure inference unit 22, and the prognosis input from the prognosis inference unit 23, and outputs the generated display data to the display device 2.

In the configuration described above, the interface 13 is an example of an image acquisition means, the lesion diagnosis unit 21 is an example of a lesion diagnosis means, the surgical procedure inference unit 22 is an example of a surgical procedure inference means, the prognosis inference unit 23 is an example of a prognosis inference means, and the output unit 24 is an example of an output means.

Note that the information processing apparatus 1 may be configured as a single unit combining the lesion diagnosis unit 21, the surgical procedure inference unit 22, and the prognosis inference unit 23. For instance, the information processing apparatus 1 may be configured by combining the surgical procedure inference unit 22 and the prognosis inference unit 23. The combination of the surgical procedure inference unit 22 and the prognosis inference unit 23 is also referred to as a “surgical procedure and prognosis inference unit” hereinafter. The surgical procedure and prognosis inference unit estimates the recommended surgical procedure and the prognosis inference based on the endoscopic images input from the interface 13 and the diagnostic information input from the lesion diagnosis unit 21. In detail, the surgical procedure and prognosis inference unit can estimate the recommended surgical procedure and the prognosis based on the endoscopic images and the diagnostic information using a surgical procedure and prognosis inference model which will be described later.

### [Inference Model]

### (Surgical Inference Model)

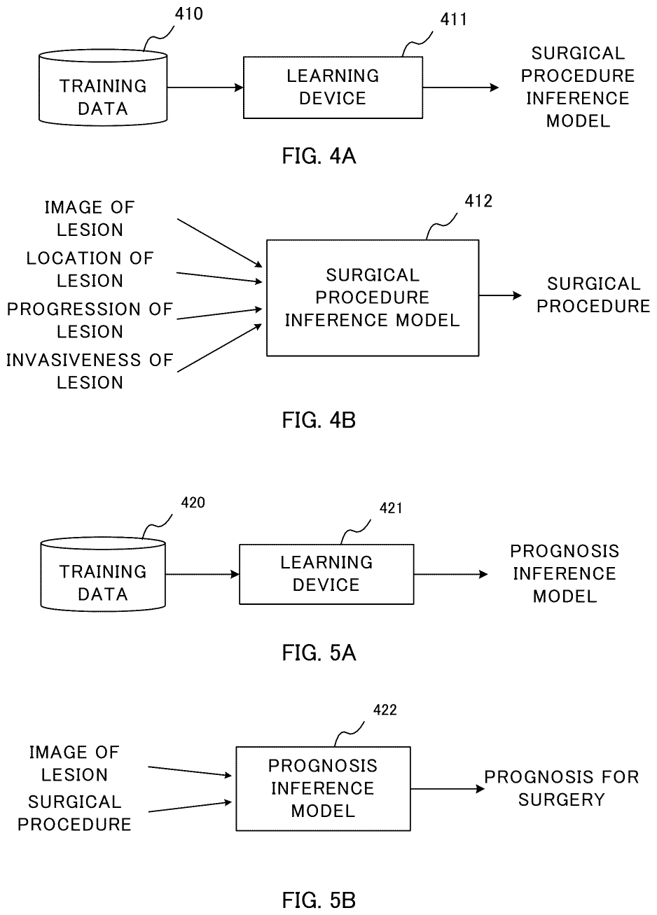

Next, the surgical procedure inference model used by the surgical procedure inference unit 22 will be described. FIG. 4A is a block diagram illustrating a learning method of the surgical procedure inference model. The surgical procedure inference model is generated by so-called supervised learning. In FIG. 4A, training data 410 and a learning device 411 are illustrated. The training data 410 are data representing a relationship between the diagnostic information of the lesion (the site of the lesion, the degree of progression, and the degree of invasion) and the lesion images, and the recommended surgical procedure for the lesion. The learning device 411 generates, based on the training data 410, the surgical procedure inference model that learns the relationship between the lesion images and the diagnostic information of the lesion and the recommended surgical procedure for the lesion. FIG. 4B is a block diagram illustrating a relationship between input data and output data of the surgical procedure inference model. A surgical procedure inference model 412 outputs the recommended surgical procedure by inputting the lesion images and the diagnostic information of the lesion.

In the above, the lesion images and the diagnostic information of the lesion images are used as input information, but it is also possible to generate the surgical procedure inference model capable of estimating the recommended surgical procedure even in a case where the input information is partially insufficient.

For instance, the learning device 411 may generate the surgical procedure inference model that learns the relationship between the lesion images and a portion of the diagnostic information of the lesion (one or more of the degree of progression and degree of invasion) and the recommended surgical procedure for the lesion. In this case, the surgical procedure inference model can output the recommended surgical procedure by inputting the lesion images and the portion of the diagnostic information of the lesion (one or more of the degree of progression and the degree of invasion).

Also, the learning device 411 can generate the surgical procedure inference model that learns the relationship between the portion of the diagnostic information of the lesion (one or more of the degree of progression and the degree of invasion of the lesion) and the recommended surgical procedure for the lesion. In this case, the surgical procedure inference model can output the recommended surgical procedure only by inputting the portion of the diagnostic information of the lesion (one or more of the degree of progression and the degree of invasion). Moreover, the learning device 411 can generate the surgical procedure inference model that learns the relationship between the lesion images and the recommended surgical procedure for the lesion. In this case, the surgical procedure inference model can output the recommended surgical procedure by inputting only the lesion images.

### (Prognosis Inference Model)

Next, the prognosis inference model used by the prognosis inference unit 23 will be described. FIG. 5A is a block diagram showing a learning method of the prognosis inference model. The prognosis inference model is generated by so-called supervised learning. FIG. 5A includes training data 420 and a learning device 421. The training data 420 are data showing the relationship between the lesion images and the recommended surgical procedure for the lesion and the prognosis. The learning device 421 generates the prognosis inference model that learns the relationship between the lesion images and the recommended surgical procedure for the lesion and the prognosis, based on the training data 420. FIG. 5B is a block diagram showing the relationship between input data and the output data for the prognosis inference model. The prognostic inference model 422 outputs the prognosis by inputting the lesion images and the recommended surgical procedure for the lesion.

Note that in the above description, the lesion images and the recommended surgical procedure for the lesion are used as input information, but it is also possible to generate a prognosis model that can estimate the prognosis even in a case where the input information is partially insufficient. For instance, the learning device 421 may generate a prognostic inference model that learns a relationship between a recommended surgical procedure for a lesion subject and a prognosis. In this case, the prognosis inference model can output the prognosis by inputting only the recommended surgical procedure for the lesion.

### (Surgical Procedure and Prognostic Inference Model)

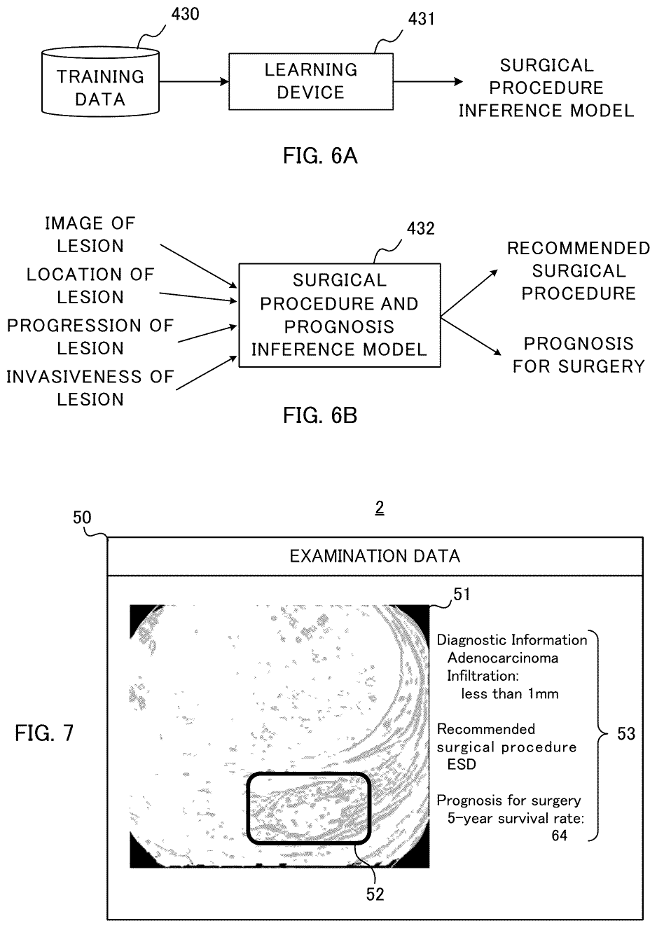

Next, the surgical procedure and prognosis inference model used by the surgical procedure and prognosis inference unit will be described. FIG. 6A is a block diagram illustrating a learning method of the surgical procedure and prognosis inference model. The surgical procedure and prognosis inference model is generated by so-called supervised learning. FIG. 6A includes training data 430 and a learning device 431. The training data 430 are data representing a relationship between a set including the lesion images and the diagnostic information of the lesion (the site of the lesion, the degree of progression, and the degree of invasion) and a set including the recommended surgical procedure for the lesion and the prognosis. The learning device 431 generates the surgical procedure and prognosis inference model that learns the relationship between the set including the lesion images and the diagnostic information of the lesion and the set including the recommended surgical procedure and the prognosis, based on the training data 430. FIG. 6B is a block diagram showing the relationship between input data and output data for the surgical procedure and prognosis inference model. The surgical procedure and prognosis inference model 432 outputs the recommended surgical procedure and the prognosis by inputting the lesion images and the diagnostic information of the lesion.

In the above description, the lesion images and the diagnostic information of the lesion are used as input information, but it is also possible to generate the recommended surgical procedure and prognosis inference mode that can estimate the surgical procedure and the prognosis even if the input information is partially insufficient.

For instance, the learning device 431 can generate the surgical procedure and prognosis inference model that learns the relationship between the set including the lesion images and the portion of the diagnostic information of the lesion (one or more of the degree of progression and the degree of invasion) and the set including the recommended surgical procedure for the lesion and the prognosis. In this case, the surgical procedure and prognosis inference model can output the recommended surgical procedure and the prognosis by inputting the lesion images and the portion of the diagnostic information of the lesion (one or more of the degree of progression and the degree of invasion).

Moreover, the learning device 431 can generate the surgical procedure and prognosis inference model which learns the relationship between the portion of the diagnostic information of the lesion (one or more of the degree of progression and the degree of invasion) and the recommended surgical procedure for the lesion and the prognosis. In this case, the surgical procedure and prognosis inference model can estimate the recommended surgical procedure and the prognosis by inputting only the portion of the diagnostic information of the lesion (one or more of the degree of progression and the degree of invasion). Furthermore, the learning device 431 can generate the surgical procedure and prognosis inference model that learns the relationship between the lesion images and a set including the recommended surgical procedure for the lesion and the prognosis. In this case, the surgical procedure prognosis inference model can output the recommended surgical procedure and the prognosis only by inputting the lesion images.

### Display Examples

Next, display examples by the display device 2 will be described.

FIG. 7 is the display example by the display device 2. Referring to the display example in FIG. 7, in a display area 50, an endoscopic image 51, a lesion region 52, and relevant information 53 are displayed. The endoscopic image 51 is the endoscopic video Ic during the endoscopic examination. The lesion region 52 is represented by a rectangle surrounding a region of the detected lesion. The relevant information 53 is information concerning the detected lesion, and includes the diagnostic information, the recommended surgical procedure and information concerning the prognosis. By viewing the relevant information 53, the doctor can ascertain the recommended surgical procedure and the prognosis of the detected lesion.

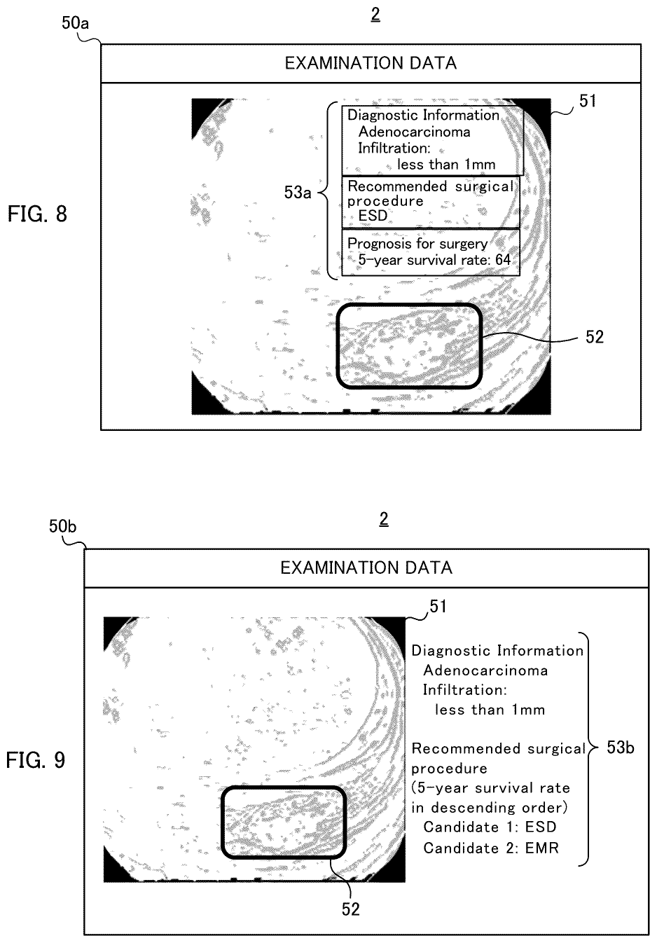

FIG. 8 shows another display example by the display device 2. Referring to the display example shown in FIG. 8, in a display area 50a, relevant information 53a is superimposed on the endoscopic image 51. The diagnostic information, the recommended surgical procedure, and the prognosis included in the relevant information 53a may be displayed collectively, or may be displayed one by one in order. For instance, the diagnostic information may be displayed upon pressing a button of the input unit 14 once by the doctor, the recommended surgical procedure may be displayed upon pressing the button of the input unit 14 twice by the doctor, and the prognosis may be displayed upon pressing the button of the input unit 14 three times by the doctor.

FIG. 9 shows another display example by the display device 2. This example is a display example in a case where several recommended surgical procedures are output for a single lesion. Referring to the display example in FIG. 9, in a display area 50b, two types of recommended surgical procedures are included in relevant information 53b, and the information processing apparatus 1 displays the recommended surgical procedures sorted by the five-year survival rate. A sorting rule is not limited to an order of the five-year survival rate, and can be set by the doctor.

Incidentally, in FIG. 9, although two types of recommended surgical procedures are displayed, in a case where three or more types of recommended surgical procedures are output for the single lesion, three or more types of recommended surgical procedures may be displayed depending on a display space. Moreover, in a case where several recommended surgical procedures are output for the single lesion and all the recommended surgical procedures cannot be displayed, the recommended surgical procedure with a good prognosis may be displayed in priority, and the recommended surgical procedure ranked higher during sorting may be displayed in priority.

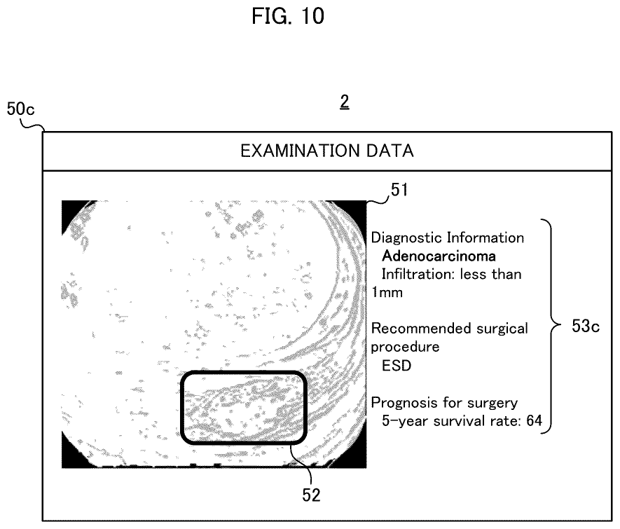

FIG. 10 shows another display example by the display device 2. This example is an example where a rationale for outputting the recommended surgical procedure is shown. In the example of FIG. 10, the information underlying the recommended surgical procedure is highlighted in bold or underlined. Specifically, in the example in FIG. 10, the recommended surgical procedure is ESD, and “adenocarcinoma” included in the diagnostic information are highlighted in bold or underlined. This indicates that the rationale for using ESD as the recommended surgical procedure is “adenocarcinoma”. Although FIG. 10 shows the rationale of the recommended method, the rationale of the diagnostic information or the rationale of the prognosis may be indicated. In this case, the rationale of the diagnostic information, the rationale of the recommended surgical procedure, and the rationale of the prognosis may be displayed at the same time in a different display mode, or only any of the rationales may be displayed based on an instruction of the doctor.

### [Image Display Process]

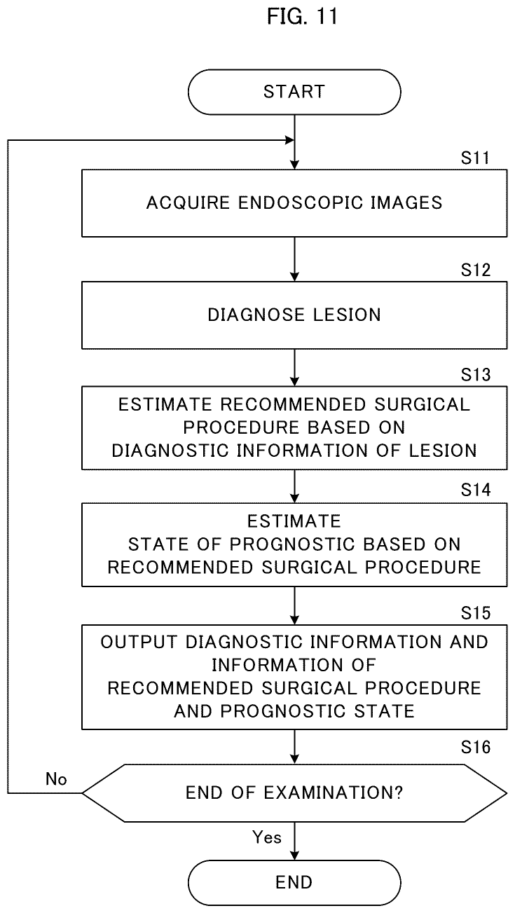

Next, an image display process for performing the above-described display. FIG. 11 is a flowchart of an image display process by the information processing apparatus 1. This process is realized by the processor 11 shown in FIG. 2 executing a corresponding program prepared in advance and operating as each element shown in FIG. 3.

First, an endoscopic video Ic is input from the endoscope scope 3 to the information processing apparatus 1. The endoscopic video Ic is input to the interface 13. The interface 13 extracts each endoscopic image from the input endoscopic video Ic, and outputs the extracted endoscopic image to the lesion diagnosis unit 21, the surgical procedure inference unit 22, and the prognosis inference unit 23. Moreover, the interface 13 outputs the endoscopic video Ic to the output unit 24 (step S11).

Next, the lesion diagnosis unit 21 detects the lesion and diagnoses the lesion based on each endoscopic image input from the interface 13. Upon detecting the lesion, the lesion diagnosis unit 21 outputs the information of the time stamp or the like and the diagnostic information to the surgical procedure inference unit 22 and the output unit 24 (step S12).

Next, the surgical procedure inference unit 22 estimates the recommended surgical procedure based on the endoscopic images input from the interface 13 and the diagnostic information input from the lesion diagnosis unit 21. The surgical procedure inference unit 22 outputs the information of the time stamp or the like and the recommended surgical procedure, to the prognosis inference unit 23 and the output unit 24 (step S13).

Next, the prognosis inference unit 23 estimates the prognosis based on endoscopic images input from the interface 13 and the recommended surgical procedure input from the surgical procedure inference unit 22. The prognosis inference unit 23 outputs the information of the time stamp or the like and the prognosis to the output unit 24 (step S14).

Next, the output unit 24 generates the display data based on the endoscopic video Ic input from the interface 13, the diagnostic information input from the lesion diagnosis unit 21, the recommended surgical procedure input from the surgical procedure inference unit 22, and the prognosis input from the prognosis inference unit 23, and outputs the generated display data to the display device 2 (step S15).

Next, the information processing apparatus 1 determines whether the endoscopic examination has been completed (step S16). For instance, the information processing apparatus 1 determines that the endoscopic examination is completed if the doctor performs an operation of an examination end with respect to the information processing apparatus 1 or the endoscope 3. Moreover, the information processing apparatus 1 may automatically determine that the endoscopic examination is complete upon detecting, through an image analysis of images captured by the endoscope 3, that a captured image shows the outside of an organ. In a case where the endoscopic examination is not complete (step S16: No), the image display process goes back to step S11. On the other hand, in a case where the endoscopic examination is complete (step S16: Yes), the image display process is terminated.

### [Modifications]

Next, modifications of the first example embodiment will be described. The following modifications can be combined as appropriate and applied to the first embodiment.

### (Modification 1)

In the first example embodiment, the output unit 24 generates the display data including the diagnostic information, the recommended surgical procedure, and the prognosis, and outputs the generated display data to the display device 2. Alternatively, the output unit 24 may generate audio data including the diagnostic information, the recommended surgical procedure, and the prognosis, and output it to the sound output unit 16. Accordingly, it is possible for the doctor to ascertain the diagnostic information, the recommended surgical procedure, and the prognosis without the endoscopic video being obstructed by other displays.

### (Modification 2)

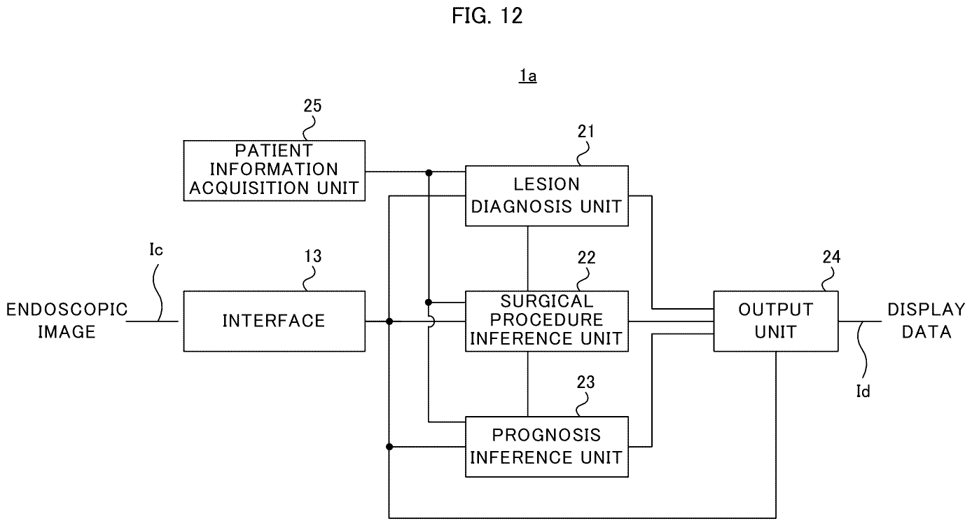

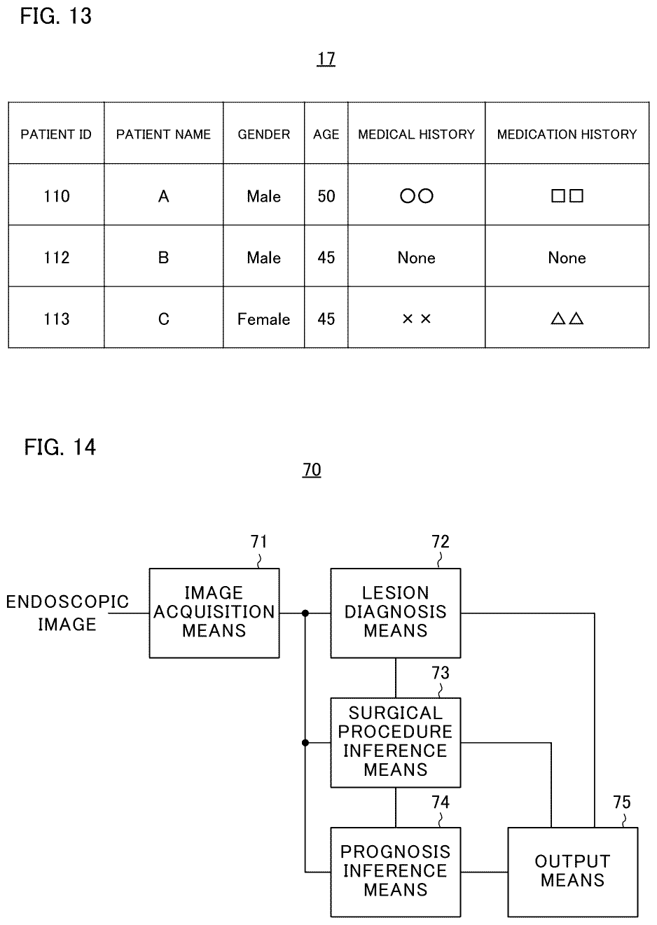

The lesion diagnosis unit 21, the surgical procedure inference unit 22, and the prognosis inference unit 23 of the first example embodiment can improve accuracy of an estimation process by considering the patient information. FIG. 12 shows a functional configuration of an information processing apparatus 1a according to a modification 2. As shown in FIG. 12, the information processing apparatus 1a is provided with a patient information acquisition unit 25. The patient information acquisition unit 25 acquires the patient information from the DB 17. FIG. 13 shows an example of a data structure of the patient information. The patient information includes information such as a patient ID, a patient name, a gender, an age, a medical history, and a medication history.

Returning to FIG. 12, the patient information acquisition unit 25 outputs the acquired patient information to the lesion diagnosis unit 21, the surgical procedure inference unit 22, and the prognosis inference unit 23.

The lesion diagnosis unit 21 detects the lesion and diagnoses the lesion, based on the endoscopic image input from the interface 13 and the patient information input from the patient information acquisition unit 25. For instance, in a case of detecting a protrusion in the endoscopic images, the lesion diagnosis unit 21 can estimate aa likelihood of a protrusion being a lesion based on whether a patient has a medical history of conditions such as colitis. Incidentally, the lesion diagnosis model used by the lesion diagnosis unit 21 is a trained model which has been trained so as to detects the lesion and diagnoses the lesion based on the endoscopic images and the patient information.

The surgical procedure inference unit 22 estimates the recommended surgical procedure based on the endoscopic images input from the interface 13, the diagnostic information input from the lesion diagnosis unit 21, and the patient information input from the patient information acquisition unit 25. For instance, the surgical procedure inference unit 22 can estimate the recommended surgical procedure suitable for an individual patient based on the age of the patient, the medication history, and the like. The surgical procedure inference model used by the surgical procedure inference unit 22 is a trained model which has been trained in advance so as to estimate the recommended surgical procedure based on the lesion images, the diagnostic information of the lesion, and the patient information.

The prognosis inference unit 23 estimates the prognosis based on the endoscopic image input from the interface 13, the recommended surgical procedure input from the surgical procedure inference unit 22, and the patient information input from the patient information acquisition unit 25. The prognosis inference model used by the prognosis inference unit 23 is a trained model which has been trained in advance so as to estimate the prognosis based on the lesion images, the recommended surgical procedure for the lesion, and the patient information.

### (Modification 3)

The lesion diagnosis unit 21 is diagnosed lesion based on the endoscopic image input from the interface 13. Alternatively, the lesion diagnosis unit 21 may diagnose the lesion based on the image from which a lesion region is cropped.

For instance, in a case where the lesion diagnosis unit 21 detects the lesion in an endoscopic image, the lesion diagnosis unit 21 crops a predetermined region (lesion region) including the lesion from the endoscopic image. Cropping refers to extracting a portion of an image. Then, the lesion diagnosis unit 21 resizes the image cropped the lesion region to a size capable of the image analysis by the lesion diagnosis model. The lesion diagnosis unit 21 diagnoses the lesion based on the re-sized image (hereinafter, also referred to as a “lesion region image”). Also, for the surgical procedure inference unit 22 and the prognosis inference unit 23, instead of the endoscopic images, the recommended surgical procedure or the prognosis may be estimated based on the lesion region image. Accordingly, by performing preprocessing of the endoscopic image, it is possible to improve the accuracy of the estimation process.

### (Modification 4)

In a case where there are a plurality of endoscopic images concerning the same lesion, the lesion diagnosis unit 21 may select an endoscopic image (hereinafter, also referred to as a “champion image”) that best represents the lesion, and diagnose the lesion based on the champion image.

For instance, in a case of detecting the lesion from the endoscopic image input from the interface 13, the lesion diagnosis unit 21 groups a plurality of endoscopic images including the endoscopic image in which the lesion is detected and endoscopic images before and after that endoscopic image. Next, the lesion diagnosis unit 21 selects the champion image from the image group where the plurality of endoscopic images are grouped. The champion image is, for instance, an image in which the lesion appears the largest, the lesion appears most centered, or the image is most in focus, among the plurality of endoscopic images which are grouped. The lesion diagnosis unit 21 diagnoses the lesion based on the champion image. Note that, the surgical procedure inference unit 22 and the prognosis inference unit 23 also may estimate the recommended surgical procedure and the prognosis based on the champion image. Accordingly, by using the champion image, it is possible to improve the accuracy of the estimation process.

### (Modification 5)

The surgical procedure inference unit 22 estimates the recommended surgical procedure based on the diagnostic information input from the lesion diagnosis unit 21. Instead, the surgical procedure inference unit 22 may estimate the recommended surgical procedure based on the diagnostic information input by the doctor. Specifically, in a case where the diagnostic information output by the lesion diagnosis unit 21 differs from findings of the doctor, the doctor inputs the diagnostic information based on his/her own opinion to the information processing apparatus 1 via the input unit 14. Then, the surgical procedure inference unit 22 estimates the recommended surgical procedure based on the diagnostic information input by the doctor. Also, the prognosis inference unit 23 may estimate the prognosis based on the recommended surgical procedure input by the doctor, instead of the recommended surgical procedure input from the surgical procedure inference unit 22. Accordingly, it is possible for the information processing apparatus 1 to also estimate the recommended surgical procedure and the prognosis with the findings of the doctor.

### (Modification 6)

In addition to the display device 2 of the first example embodiment, a display device for a patient (hereinafter, also referred to as a “patient monitor”) may be provided. The patient monitor is used for the patient to view the endoscopic examination. Although only endoscopic images are basically displayed on the monitor for the patient, it is also possible to display information specified by the doctor. For instance, in a case where the lesion is detected during the endoscopic examination, the doctor can cause the patient monitor to display the diagnostic information or the like by a predetermined operation. In addition, the information to be output to the patient monitor can be controlled using a predetermined condition or a machine learning model. Accordingly, it is possible for the doctor to provide explanations that are easy for the patient to understand.

### Second Example Embodiment

FIG. 14 is a block diagram illustrating a functional configuration of an information processing apparatus according to a second example embodiment. The information processing apparatus 70 includes an image acquisition means 71, a lesion diagnosis means 72, a surgical procedure inference means 73, a prognosis inference means 74, and an output means 75.

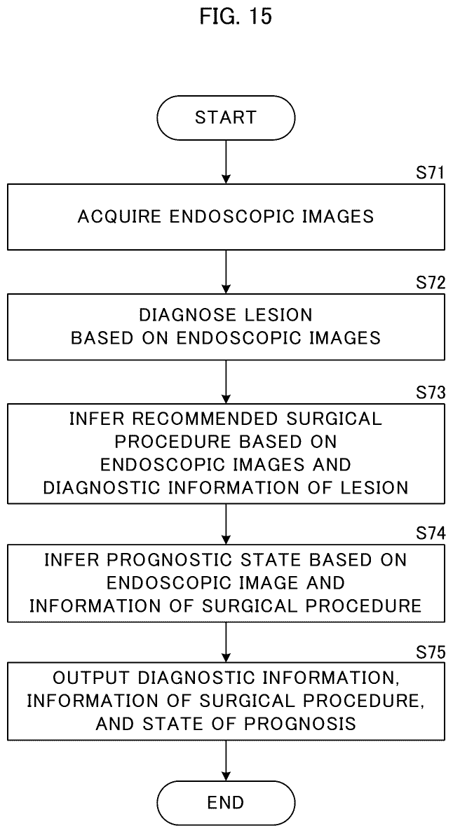

FIG. 15 is a flowchart of processing performed by the information processing apparatus according to the second example embodiment. The image acquisition means 71 acquires each endoscopic image captured by the endoscope (step S71). The lesion diagnosis means 72 diagnoses the lesion based one or more endoscopic images (step S72). The surgical procedure inference means 73 infers the recommended surgical procedure based on the endoscopic images and the diagnostic information of the lesion (step S73). The prognosis inference means 74 infers a state of the prognosis based on the endoscopic images and information of the surgical procedure (step S74). The output means 75 outputs the diagnostic information, the information of the surgical procedure, and the state of the prognosis (step S75).

According to the information processing apparatus 70 of the second example embodiment, it becomes possible to estimate the recommended surgical procedure and the prognosis for the lesion found in the endoscopic examination.

A part or all of the example embodiments described above may also be described as the following supplementary notes, but not limited thereto.

### (Supplementary Note 1)

An information processing apparatus comprising:
an image acquisition means configured to acquire endoscopic images captured by an endoscope;
a lesion diagnosis means configured to diagnose a lesion based on the endoscopic images;
a surgical procedure inference means configured to infer a surgical procedure which is recommended based on the endoscopic images and diagnosis information of the lesion;
a prognosis inference means configured to infer a state of a prognosis based on the endoscopic images and information of the surgical procedure; and
an output means configured to output the diagnosis information, the information of the surgical procedure, and the state of the prognosis.

### (Supplementary Note 2)

The information processing apparatus according to supplementary note 1, wherein the output means generates image data based on the diagnosis information of the lesion, the information of the surgical procedure, and the state of the prognosis, and outputs the image data to a display device.

### (Supplementary Note 3)

The information processing apparatus according to supplementary note 2, wherein the output means generates display data including a rationale of the diagnosis information of the lesion, a rationale for inferring the surgical procedure, and a rationale for inferring the state of the prognosis, and outputs the display data to the display device.

### (Supplementary Note 4)

The information processing apparatus according to supplementary note 1, wherein in a case where there are several surgical procedures, the output means sorts the several surgical procedures based on respective states of the prognosis.

### (Supplementary Note 5)

The information processing apparatus according to supplementary note 1, further comprising a selection means configured to select a champion image that best represents the lesion from among a plurality of endoscopic images corresponding to the same region, wherein
the lesion diagnosis means diagnoses the lesion based on the champion image,
the surgical procedure inference means infers the surgical procedure which is recommended based on the champion image and the diagnosis information of the lesion, and
the prognosis inference means infers the state of the prognosis based on the champion image and the information of the surgical procedure.

### (Supplementary Note 6)

The information processing apparatus according to supplementary note 1, further comprising a patient information acquisition means configured to acquire patient information, wherein
the lesion diagnosis means diagnoses the lesion based on the endoscopic images and the patient information,
the surgical procedure inference means infers the surgical procedure which is recommended based on the endoscopic images, the diagnosis information of the lesion, and the patient information, and
the prognosis inference means infers the state of the prognosis based on the endoscopic images, the diagnosis information of the lesion, and the patient information.

### (Supplementary Note 7)

An information processing method comprising:
acquiring endoscopic images captured by an endoscope;
diagnosing a lesion based on the endoscopic images;
inferring a surgical procedure which is recommended based on the endoscopic images and diagnosis information of the lesion;
inferring a state of a prognosis based on the endoscopic images and information of the surgical procedure; and
outputting the diagnosis information, the information of the surgical procedure, and the state of the prognosis.

### (Supplementary Note 8)

A recording medium storing a program, the program causing a computer to perform a process comprising:
acquiring endoscopic images captured by an endoscope;
diagnosing a lesion based on the endoscopic images;
inferring a surgical procedure which is recommended based on the endoscopic images and diagnosis information of the lesion;
inferring a state of a prognosis based on the endoscopic images and information of the surgical procedure; and
outputting the diagnosis information, the information of the surgical procedure, and the state of the prognosis.

While the disclosure has been described with reference to the example embodiments and examples, the disclosure is not limited to the above example embodiments and examples. It will be understood by those of ordinary skill in the art that various changes in form and details may be made therein without departing from the spirit and scope of the present disclosure as defined by the claims.

### DESCRIPTION OF SYMBOLS

1 Information processing apparatus
2 Display device
3 Endoscope
11 Processor
12 Memory
13 Interface
17 Database (DB)
21 Lesion diagnosis unit
22 Surgical procedure inference unit
23 Prognosis inference unit
24 Output unit
100 Information processing system

What is claimed is:

1. An image processing apparatus for endoscopic examination comprising:
at least one memory configured to store instructions; and
at least one processor configured to execute the instructions to:
acquire endoscopic images captured by an endoscope;
diagnose a lesion based on the endoscopic images;
infer a surgical procedure which is recommended based on the endoscopic images and diagnosis information of the lesion by using surgical inference model trained by machine learning with training data indicating a relationship between the diagnostic information of the lesion, lesion images and recommended surgical procedure for the lesion;
infer a state of a prognosis based on the endoscopic images and information of the surgical procedure by using prognosis inference model trained by machine learning with training data indicating a relationship between the lesion images, the recommended surgical procedure for the lesion and the state of the prognosis; and
output the diagnosis information, the information of the surgical procedure, and the state of the prognosis.

2. The image processing apparatus according to claim 1, wherein the one or more processors generate image data based on the diagnosis information of the lesion, the information of the surgical procedure, and the state of the prognosis, and outputs the image data to a display device.

3. The image processing apparatus according to claim 2, wherein the one or more processors generate display data including a rationale of the diagnosis information of the lesion, a rationale for inferring the surgical procedure, and a rationale for inferring the state of the prognosis, and outputs the display data to the display device.

4. The image processing apparatus according to claim 1, wherein in a case where there are several surgical procedures, the one or more processors sort the several surgical procedures based on respective states of the prognosis.

5. The image processing apparatus according to claim 1, wherein the one or more processors select a champion image that best represents the lesion from among a plurality of endoscopic images corresponding to the same region, wherein
the one or more processors diagnose the lesion based on the champion image,
the one or more processors infer the surgical procedure which is recommended based on the champion image and the diagnosis information of the lesion, and
the one or more processors infer the state of the prognosis based on the champion image and the information of the surgical procedure.

6. The image processing apparatus according to claim 1, wherein the one or more processors further acquire patient information,
wherein one or more processors
the diagnose the lesion based on the endoscopic images and the patient information,
the infer the surgical procedure which is recommended based on the endoscopic images, the diagnosis information of the lesion, and the patient information, and
the infer the state of the prognosis based on the endoscopic images, the diagnosis information of the lesion, and the patient information.

7. An image processing method for endoscopic examination comprising:
acquiring endoscopic images captured by an endoscope;
diagnosing a lesion based on the endoscopic images;
inferring a surgical procedure which is recommended based on the endoscopic images and diagnosis information of the lesion by using surgical inference model trained by machine learning with training data indicating a relationship between the diagnostic information of the lesion, lesion images and recommended surgical procedure for the lesion;
inferring a state of a prognosis based on the endoscopic images and information of the surgical procedure by using prognosis inference model trained by machine learning with training data indicating a relationship between the lesion images, the recommended surgical procedure for the lesion and the state of the prognosis; and
outputting the diagnosis information, the information of the surgical procedure, and the state of the prognosis.

8. A non-transitory computer readable recording medium storing a program, the program causing a computer to perform a process comprising:
acquiring endoscopic images captured by an endoscope;
diagnosing a lesion based on the endoscopic images;
inferring a surgical procedure which is recommended based on the endoscopic images and diagnosis information of the lesion by using surgical inference model trained by machine learning with training data indicating a relationship between the diagnostic information of the lesion, lesion images and recommended surgical procedure for the lesion;
inferring a state of a prognosis based on the endoscopic images and information of the surgical procedure by using prognosis inference model trained by machine learning with training data indicating a relationship between the lesion images, the recommended surgical procedure for the lesion and the state of the prognosis; and
outputting the diagnosis information, the information of the surgical procedure, and the state of the prognosis.
